SEQUENCE LISTING (4 sequences)

SEQ ID NO: 1 - DNA, 24 bp, Artificial Sequence
  Synthetic IL-6 5′ primer
atgaagttcctctctgcaagagac

SEQ ID NO: 2 - DNA, 24 bp, Artificial Sequence
  Synthetic IL-6 3′ primer
cactaggtttgccgagtagatctc

SEQ ID NO: 3 - DNA, 20 bp, Artificial Sequence
  Synthetic Beta actin 5′ primer
gtggggcgccccaggcacca

SEQ ID NO: 4 - DNA, 24 bp, Artificial Sequence
  Synthetic beta actin 3′ primer
ctccttaatgtcacgcacgatttc

(19) United States
(12) Patent Application Publication
(10) Pub. No.: US 2011/0135672 A1
(43) Pub. Date: Jun. 9, 2011

(54) HLA-G COMPOSITIONS AND METHODS OF USE THEREOF

(75) Inventors: Anatolij Horuzsko, Evans, GA (US); Joseph Kaminski, Evans, GA (US)

(73) Assignee: Medical College of Georgia Research Institute, Inc. Georgia

(21) Appl. No.: 12/788,623

(22) Filed: May 27, 2010

Related U.S. Application Data
(63) Continuation of application No. PCT/US2009/033084, filed on Feb. 4, 2009.
(60) Provisional application No. 61/063,316, filed on Feb. 4, 2008.
(60) Provisional application No. 61/063,314, filed on Feb. 4, 2008.

(51) Int. Cl. A61K 39/00; A61P 37/06; C07K 14/435; C07K 16/00; B32B 5/00; C12N 5/00; B82Y 5/00

(52) U.S. Cl. 424/185.1; 530/350; 530/391.1; 428/402; 435/375; 977/773

(57) ABSTRACT

It has been discovered that HLA-G dimers are potent ligands of ILT2 and ILT4 on immune cells and enhance signal transduction through ILT2 and ILT4. Enhanced signal transduction through ILT-2, ILT-4 or both down-regulates the biological activity of T cells and dendritic cells. HLA-G compositions including HLA-G dimers are provided that are useful for modulating activity of immune cells. Preferred compositions include microparticles having HLA-G dimers on the surface of the microparticles. The microparticles optionally include a targeting moiety to target the microparticles to specific immune cells. In a preferred embodiment the microparticles are targeted to T cells or dendritic cells expressing ILT2 or both ILT2 and ILT4, respectively. The HLA-G dimer can include any HLA-G protein that is capable of forming a dimer. Preferred HLA-G proteins include HLA-G1 and HLA-G5. In certain embodiments the microparticles include dimers of HLA-G1 and HLA-G5.

### CROSS REFERENCE TO RELATED APPLICATIONS

This application claims benefit of and priority to U.S. Provisional Patent Application No. 61/063,316 filed on Feb. 4, 2008, and to U.S. Provisional Patent Application No. 61/063,314 also filed on Feb. 4, 2008.

### STATEMENT REGARDING FEDERALLY SPONSORED RESEARCH OR DEVELOPMENT

This invention was made with Government Support under Agreement NIH A1055923 award to Anatolij Horuzsko by the National Institutes of Health. The Government has certain rights in the invention.

### FIELD OF THE INVENTION

The invention is generally related to the field of immunology, in particular to HLA-G compositions for modulating immunocompetent cells and methods for treating inflammatory disorders, autoimmune disorders and graft rejection.

### BACKGROUND OF THE INVENTION

Organ transplantation has demonstrated both a survival and quality of life benefit for selected patients with end-stage disease. The development of immunosuppressive therapies has led to remarkable success of clinical allotransplantation. Despite improvements in early survival, however, survival beyond 5 years remains poor. The initial step of immunological response directed towards engrafted organ is antigen recognition that could have either a direct or indirect pattern. Direct recognition pattern initiates acute graft rejection. In contrast, processed donor antigens presented with MHC on recipient antigen presenting cells (APCs) are engaged in indirect recognition pattern, which is more important in chronic graft rejection (Sayegh, M. H., et al., Int Rev Immunol, 13:221 (1996); Benichou, G., et al., J Immunol, 162:352 (1999)). A key factor driving the chronic rejection in clinical transplantation is a persistent T cell-mediated alloimmune response (Clarkson, M. R., et al., Transplantation, 80:555 (2005)). Therefore, the development of new therapeutic strategies to target T cell alloimmune responses is an important direction to prevent and manage chronic rejection.

A variety of peripheral tolerance mechanisms ensure that cells escaping negative selection in the thymus are prevented from causing autoimmunity. These mechanisms can broadly be summarized as leading to immunological ignorance, deletion, or suppression. The successful semiallogeneic pregnancy is the natural model where multiple mechanisms underlie maternal tolerance of genetically different fetal tissues during pregnancy. The fetal contributions to the maternal tolerance are unique and mostly based on fetal-derived tissues, trophoblast cells. The antigens expressed on trophoblast cells program maternal immunocompetent cells into pathways consistent with tolerance. The key player in this process on trophoblast cells is HLA-G, an unique protein that remains as an antigen of great interest and a focus of experimental evaluation.

### HLA-G and its Inhibitory Receptors.

The ability of cell-surface receptors and their ligands to counterregulate innate and adaptive components of the inflammatory process is a relatively new concept in the field of transplantation immunology. The inflammatory responses contribute a substantial role to rejection of allografts, especially in early stages of posttransplantation, when a large number of alloresponsive T cells are stimulated by graft antigens presented by either donor or host APCs. The mechanisms that hold the inflammatory process in check will reduce the incidence of the acute graft rejection. Therefore, the development of strategies to control the inflammatory process is a perspective way to minimize rejection of allografts.

Therefore, it is an object of the invention to provide compositions for treating one or more symptoms of an inflammatory disorder, an autoimmune disease or graft rejection.

It is another object of the invention to provide compositions for modulating immunocompetent cells.

It is yet another object of the invention to provide methods of inducing T cell tolerance.

### SUMMARY OF THE INVENTION

It has been discovered that HLA-G dimers are potent ligands of ILT2 and ILT4 on immune cells and enhance signal transduction through ILT2 and ILT4. Enhanced signal transduction through ILT-2, ILT-4 or both down-regulates the biological activity of T cells and dendritic cells. HLA-G compositions including HLA-G dimers are provided that are useful for modulating activity of immune cells. Preferred compositions include microparticles having HLA-G dimers on the surface of the microparticles. The microparticles optionally include a targeting moiety to target the microparticles to specific immune cells. In a preferred embodiment the microparticles are targeted to T cells or dendritic cells expressing ILT2 or both ILT2 and ILT4, respectively.

The HLA-G dimer can include any HLA-G protein that is capable of forming a dialer. Preferred HLA-G proteins include HLA-G1 and HLA-G5. In certain embodiments the microparticles include dimers of HLA-G1 and HLA-G5.

In one embodiment the targeting moiety is an antibody or antigen binding fragment thereof or a ligand specific for a protein displayed on the immune cell. A preferred protein for targeting dendritic cells is CD11c. The microparticle can be coated with CD11c or a fragment thereof that is bound by an antibody or antigen binding fragment thereof. Ligands specific for immune cells or coupling agents for attaching ligands of immune cells are typically present in high density. For example, the ligands can be present in a range of 1,000 to 10,000,000 ligands per square micron of microparticle surface area.

In another embodiment, the microparticle is polymeric. Although a variety of polymers and copolymers can be used to manufacture the microparticles, biodegradable polymers are preferred. The microparticles can have a diameter of 0.5 to 1000 microns. In certain embodiments the microparticles have a diameter of 50 nm to 500 nanometers.

The microparticle optionally includes one or more therapeutic agents. Exemplary therapeutic agents are anti-inflammatory agents. In other embodiments, the microparticles include a physiologically or pharmaceutically acceptable carrier, excipient, or stabilizer.

Still another embodiment provides a method for delaying transplant rejection or increasing T cell tolerance in a subject by administering an effective amount of the disclosed microparticles to the subject to enhance signal transduction through ILT2 or ILT4 on immune cells relative to a control.

Other methods for using the microparticles include treating one or more symptoms of an inflammatory disorder in a subject by administering an effective amount of the disclosed microparticles, treating one or more symptoms of an autoimmune disorder in a subject by administering an effective amount of the disclosed microparticles, and decreasing the level of expression of MHC class II, CD80, CD86 or a combination thereof in dendritic cells by contacting the dendritic cells with an effective amount of the disclosed microparticles.

### BRIEF DESCRIPTION OF THE DRAWINGS

FIG. 1A shows flow cytometry graphs of bone marrow-derived DC (BMDC) obtained from ILT4 transgenic mice and treated with microparticles coated with HLA-G tetramer complexes or microparticle coated with both HLA-G tetramer complexes and purified anti-CD11c mAb. FIG. 1B shows panels of flow cytometry histograms of immature BMDC treated with microparticles coated with HLA-G tetramer complexes or microparticle coated with both HLA-G tetramer complexes and purified anti-CD11c mAb. FIG. 1C shows panels of flow cytometry histograms C) demonstrating the effect of single (HLA-G1-coated) and double (HLA-G1 and anti-CD11c mAb-coated) microparticles on activation/maturation of ILT4-positive DC in vivo induced by allogeneic skin transplant.

FIG. 2A shows a gel filtration chromatograph of HLA-G5 indicating absorbance (mAU) as a function of elution volume (ml). FIG. 2B shows a panel of graphs of GFP expression on reporter cells analyzed by flow cytometry. NFAT-GFP reporter cells expressing the ILT2-PILRβ chimera were stimulated with the indicated concentration of immobilized HLA-G5m, HLA-G5d, or HLA-G1t for 18 h. Numbers indicate the percentage of GFP-positive cells (Upper) and MFI of GFP (Lower).

FIGS. 3A and 3B show panels of flow cytometry histograms for regional draining lymph node cells were analyzed on day 3 after transplantation by staining with anti-CD11c, anti-MHC class II (anti-I-A^{b}), anti-CD86, and anti-CD80 mAbs (thick line histogram) and isotype control (thin line histogram). Recipient ILT4 mice were injected with the same amount (20 ng/mice) of HLA-G5m, HLA-G5d, or HLA-G1t 24 h before allogeneic skin transplantation from MHC class II-disparate mutant bm12 donor mice. (FIG. 3A). Histograms shown were gated on the CD11c⁺ population. In FIG. 3B the permeabilized cells were stained with PE-conjugated anti-IL-6, anti-IL-10, and anti-IL-12 mAbs (thick line histogram) and isotype control (thin line histogram). Histograms shown were gated on the CD11c⁺ population. Numbers indicate percentage of total gated cells falling into selected quadrants. Data are representative of three independent experiments

FIG. 4A shows a bar graph showing fold increase in the levels of IL-6 transcription in HLA-G5m (monomer), HLA-G5d (dimer), HLA-Gt (tetramer)-treated cells. FIG. 4B shows a bar graph of RT-PCR analysis of IL-6 mRNA levels in ILT4-positive BMDCs treated with HLA-G1t and untreated cells following stimulation with 100 ng/ml of LPS. Data represent three independent experiments.

FIG. 5 shows panels of flow cytometry histograms of 2.5×10⁵ BMDCs from ILT4 transgenic mice were added to 96-well plates uncoated (first two columns) or coated with 50 ng/ml of HLA-G1 tetramer (last two columns). After 3 h incubation with ligand, cells were stimulated with different concentrations of LPS (100 ng/ml is shown) for an additional 18 h. Different concentrations of anti-IL-6 neutralizing antibody MP5-20F3 (20 ng/ml is shown) were added to the cells during stimulation with LPS (last column). MHC class II expression was assessed by flow cytometry using PE-conjugated anti-CD11c and FITC-conjugated anti-MHC class II (I-A^{b}) mAbs (thick line histogram) and isotype control (thin line histogram). Numbers indicate percentage of positive cells of total gated CD11c⁺ cells. The results are from one of three representative experiments.

FIG. 6 shows a bar graph of the levels of STAT3 activation in ILT4-positive BMDCs treated with HLA-G1t (hatched) for 3 h with and without stimulation with 100 ng/ml of LPS for the indicated time. ILT4-positive BMDCs (5×10⁶) were treated for 3 h with 50 ng/ml of HLA-G1t or were left untreated. DCs were stimulated for an additional 1 h with 100 ng/ml of LPS or left unstimulated.

FIGS. 7A and 7B are graphs showing percent of SHP-1 or SHP-2 levels respectively in ILT4-positive BMDCs infected with lentiviral particles expressing SHP-1 shRNA or SHP-2 shRNA. Cellular lysates were analysed by Western blot using antibodies against SHP-1, SHP-2, and actin. Actin was detected as a loading control. Bars represent a percentage of the maximum signal per non-target control band measured by semi-quantitative densitometry. Densities were calculated as an average of five measurements per band. FIG. 7C is a bar graph showing relative IL-6 mRNA in controls or SHP-1 or SHP-2 knockdowns. RNA was isolated 48 h post-infection with indicated lentiviral particles, and RT-PCR was performed using primers to detect β-actin (internal control) and IL-6. FIG. 7D shows a panel of flow cytometry histograms of the expression of MHC class II molecules on ILT4-positive BMDCs. Cells were stimulated with 100 ng/ml of LPS for 18 h or left un-stimulated (first 2 panels). Following transduction with the indicated lentiviral particles, cells were treated with HLA-G1t for 3 h and then stimulated with 100 ng/ml of LPS for 18 h (last 3 panels). Cells were stained with APC-conjugated anti-CD11c and FITC-conjugated anti-MHC class II (I-A^{b}) mAbs, Histograms shown were gated on CD11c⁺ population. The line with the peak furthest to the left represents the isotype control. Numbers indicate percentage of positive cells of total gated cells. The results are from one representative experiment of four performed.

FIG. 8 shows a diagram of a proposed model of arrest of maturation/activation of DCs via ILT4 receptor and HLA-G ligand. HLA-G induces phosphorylation of ILT4 receptor and recruitment of SHP-1 and SHP-2 phosphatases. SHP-2 enhances activation of NF-κB and downstream IL-6 production. IL-6 induces STATS activation, which decreases cystatin C level, the endogenous inhibitor of cathepsins, and enhanced cathepsin S activities. Cathepsin S decreased intracellular MHC class II αβ dimer levels, invariant chain (Ii), and H2-DM molecules levels in DCs. The moderate signal generated through TLR4 leads to modest induction of IL-12 and IL-6, therefore additionally enhancing IL-6 production.

### DETAILED DESCRIPTION OF THE INVENTION

### I. Definitions

Terms defined herein have meanings as commonly understood by a person of ordinary skill in the art. Terms such as “a”, “an” and “the” are not intended to refer to only a singular entity, but include the general class of which a specific example may be used for illustration.

The terms “individual”, “host”, “subject”, and “patient” are used interchangeably herein and includes rodents, simians, humans, mammalian farm animals, mammalian sport animals, and mammalian pets.

The term “effective amount” or “therapeutically effective amount” means a dosage sufficient to provide treatment of the inflammatory response or autoimmune disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

### II. HLA-G Compositions

HLA-G compositions, preferably HLA-G dimer compositions are provided. The disclosed HLA-G compositions are useful for modulating immunocompetent cells. In particular, the compositions are useful for triggering signal transduction through ILT2, ILT4 or both on cells of the immune system, for example T cells or dendritic cells (DC). In a preferred embodiment, HLA-G dimers are conjugated to a carrier. Suitable carriers include, but are not limited to microparticles, microspheres, nanoparticles or the like. The microparticles are useful for the treatment of inflammatory disorders including autoimmune diseases. The microparticles are also useful for delaying transplantation rejection.

A. HLA-G

HLA-G protein present on the disclosed microparticles is encoded by a nonclassical class Ib gene, and HLA-G protein possesses some unusual characteristics, including restricted expression, limited number of polymorphisms, and alternatively spliced mRNA variants. The genomic structure of HLA-G is similar to other class I genes, however HLA-G is unique in several other respects. The HLA-G gene has eight exons encoding a signal peptide (exon 1), the a1, a2, and a3 domains (exons 2, 3, and 4, respectively), the transmembrane domain (exon 5) and the intracellular domain (exons 6 and 7). The premature stop codon in exon 6 is responsible in slower turnover, prolonged expression of HLA-G at the cell surface, and the inefficient presentation of exogenous peptides (Park, B., et al., Immunity, 15:213 (2001)). Another unique future of HLA-G is that it encodes at least seven isoforms as a result of alternative splicing (HLA-G1 to HLA-G7). The full-length membrane-bound isoforms, HLA-G1, is structurally similar to other class I genes, except for the truncated cytoplasmic tail. A stop sequence in intron 4 results in two soluble isoforms, HLA-G5 and HLA-G6.

One of the major roles suggested for the HLA-G protein is inhibition of the function of T cells and DCs via inhibitory receptors ILT2 (LILRB1/CD85j) and ILT4 (LILRB2/CD85d) (Colonna, M., et al., J Exp Med, 186:1809 (1997)). In addition, the expression of HLA-G on the cell surface protects susceptible target cells from NK-mediated cytotoxicity (Pazmany, L., et al., Science, 274:792 (1996)). Several NK receptors were suggested as being able to recognize HLA-G (Mandelboim, O., et al., Proc Natl Acad Sci USA, 94:14666 (1997)), and recently KIR2□L4 (CD158d) receptor was identified. KIR2□L4 is expressed either on all NK cells (Rajagopalan, S., et al., J Exp Med, 189:1093 (1999)) derived from peripheral blood mononuclear cells or only on decidual NK cells (Ponce, M., et al., Proc Nall Acad Sci USA, 96:5674 (1999)).

The most important functional isoforms of HLA-G include b2-microglobulin-associated HLA-G1 and HLA-G5. However, the tolerogenic immunological effect of these isoforms is different and is dependent on the form (monomer, dimer) of ligands and the affinity of the ligand-receptor interaction. In a preferred embodiment, the disclosed compositions include the dimer form of HLA-G.

HLA-G has several free cysteine residues, unlike most of the other MHC class I molecules. Boyson et al., Proc Nall Acad Sci USA, 99: 16180 (2002) reported that the recombinant soluble form of HLA-G5 could form a disulfide-linked dimer with the intermolecular Cys42-Cys42 disulfide bond. In addition, the membrane-bound form of HLA-G1 can also form a disulfide-linked dimer on the cell surface of the Jeg3 cell line, which endogenously expresses HLA-G. Disulfide-linked dimer forms of HLA-G1 and HLA-G5 have been found on the cell surface of trophoblast cells as well (Apps, R., Tissue Antigens, 68:359 (2006)).

1. Dimers

Preferred microparticles have HLA-G dimers conjugated, attached, or adsorbed to the surface of the microparticle, in particular dimers of HLA-G1, HLA-G5, or a combination thereof. It will be appreciated that any HLA-G isoform that is capable of forming a dimer can be used. The dimers are present in an amount effective to trigger or enhance signal transduction through ILT2, ILT4, or a combination thereof as discussed below. A 50 ng/ml solution of dimer can be used to coat the microparticles.

HLA-G protein can be produced using standard molecular biology techniques. The nucleic acid sequence for HLA-G isoforms is known in the art. See for example GENBANK Accession No. AY359818. Once the protein is produced and purified, dimer formation is promoted by incubating HLA-G monomer at 4° C. for 3-7 days. The HLA-G dimers promote signal transduction through ILTs, in particular ILT2, ILT4, or a combination thereof.

2. ILTs

ILTs represent Ig types of activating and inhibitory receptors that are involved in regulation of immune cell activation and control the function of immune cells (Borges, L., et al., Curr Top Microbial Immunol, 244:123-136 (1999)). ILTs are categorized into three groups: (i) inhibitory, those containing a cytoplasmic immunoreceptor tyrosine-based inhibitory motif (ITIM) and transducing an inhibitory signal (ILT2, ILT3, ILT4, ILT5, and LIR8); (ii) activating, those containing a short cytoplasmic tail and a charged amino acid residue in the transmembrane domain (ILT1, ILT7, ILT8, and LIR6α) and delivering an activating signal through the cytoplasmic immunoreceptor tyrosine-based activating motif (ITAM) of the associated common γ chain of Fc receptor; and (iii) the soluble molecule ILT6 lacking the transmembrane domain. A number of recent studies have highlighted immunoregulatory roles for ILTs on the surface of antigen presenting cells (APC). ILT2, ILT3, and ILT4 receptors, the most characterized immune inhibitory receptors, are expressed predominantly on myeloid and plasmacytoid DC. ILT3 and ILT4 are upregulated by exposing immature DC to known immunosuppressive factors, including IL-10, vitamin D3, or suppressor CD8 T cells (Chang, C. C., et al., Nat Immunol, 3:237-243 (2002)). The expression of ILTs on DC is tightly controlled by inflammatory stimuli, cytokines, and growth factors, and is down-regulated following DC activation (Ju, X. S., et al., Gene, 331:159-164 (2004)). The expression of ILT2 and ILT4 receptors is highly regulated by histone acetylation, which contributes to strictly controlled gene expression exclusively in the myeloid lineage of cells (Nakajima, H., J Immunol, 171:6611-6620 (2003)).

Engagement of the inhibitory receptors ILT2 and ILT4 alters the cytokine and chemokine secretion profile of monocytes and can inhibit Fc receptor signaling (Colonna, M., et al. J Leukoc Biol, 66:375-381 (1999)). The role and function of ILT3 on DC have been precisely described by the Suciu-Foca group (Suciu-Foca, N., Int Immunopharmacol, 5:7-11 (2005)). Although the ligand for ILT3 is unknown, ILT4 is known to bind to the third domain of HLA class I molecules (HLA-A, HLA-B, HLA-C, and HLA-G), competing with CD8 for MHC class I binding (Shiroishi, M., Proc Natl Acad Sci USA, 100:8856-8861 (2003)). The preferential ligand for several inhibitory ILT receptors is HLA-G. HLA-G plays a potential role in maternal-fetal tolerance and in the mechanisms of escape of tumor cells from immune recognition and destruction (Hunt, J. S., et al., Faseb J, 19:681-693 (2005)). It is most likely that regulation of DC function by HLA-G-ILT interactions is an important pathway in the biology of DC. It has been determined that human monocyte-derived DC that highly express ILT2 and ILT4 receptors, when treated with HLA-G and stimulated with allogeneic T cells, still maintain a stable tolerogenic-like phenotype (CD80^{low}, CD86^{low}, HLA-DR^{low}) with the potential to induce T cell anergy (Ristich, V., et al., Eur J Invnunol, 35:1133-1142 (2005)). Moreover, the HLA-G interaction with DC that highly express ILT2 and ILT4 receptors resulted in down-regulation of several genes involved in the MHC class II presentation pathway. A lysosomal thiol reductase, IFN-γ inducible lysosomal thiol reductase (GILT), abundantly expressed by professional APC, was greatly reduced in HLA-G-modified DC. The repertoire of primed CD4⁺ T cells can be influenced by DC expression of GILT, as in vivo T cell responses to select antigens were reduced in animals lacking GILT after targeted gene disruption (Marie, M., et al., Science, 294:1361-1365 (2001)). The HLA-G/ILT interaction on DC interferes with the assembly and transport of MHC class II molecules to the cell surface, which might result in less efficient presentation or expression of structurally abnormal MHC class II molecules. The loading of exogenous peptides onto MHC class II molecules has been shown to be critically dependent on H2-M (HLA-DM, human). Cells from mice with a targeted mutation in the H2-M gene are unable to present intact proteins and have a markedly reduced capacity to present exogenous peptides (10-to 20 fold reduction) (Miyazaki, T., et al., Cell, 84:531-541 (1996)). It was determined that HLA-G markedly decreased the transcription of invariant chain (CD74), HLA-DMA, and HLA-DMB genes on human monocyte-derived DC highly expressing ILT inhibitory receptors (Ristich, V., et al; Eur J Immunol 35:1133-1142 (2005)).

B. Microparticles

As used herein, microparticles generally refers to both microparticles in the range of between 0.5 and 1000 microns and nanoparticles in the range of between 50 nm to less than 0.5, preferably having a diameter that is between 1 and 20 microns or having a diameter that is between 50 and 500 nanometers, respectively.

The external surface of the microparticles may be modified by conjugating a coupling agent or ligand to the surface of the microparticle. As described below, in the preferred embodiment, the coupling agent is present in high density on the surface of the microparticle.

As used herein, “high density” refers to microparticles having a high density of ligands or coupling agents, which is preferably in the range of 1,000 to 10,000,000, more preferably 10,000-1,000.000 ligands per square micron of microparticle surface area. This can be measured by fluorescence staining of dissolved particles and calibrating this fluorescence to a known amount of free fluorescent molecules in solution.

The microparticle may be further modified by attachment of one or more different molecules to the ligands or coupling agents, such as targeting molecules, attachment molecules, and/or therapeutic, nutritional, diagnostic or prophylactic agents.

A targeting molecule is a substance which will direct the microparticle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

Control over regional modification refers to the ability to selectively modify sections of a biodegradable scaffold without modifying the whole.

There have been a variety of materials used to engineer solid nanoparticles with and without surface functionality (as reviewed by Brigger et al. Adv Drug Deliv Rev 54, 631-651 (2002)). Perhaps the most widely used are the aliphatic polyesters, specifically the hydrophobic poly (lactic acid) (PLA), more hydrophilic poly (glycolic acid) PGA and their copolymers, poly (lactide-co-glycolide) (PLGA). The degradation rate of these polymers can vary from days (PGA) to months (PLA) and is easily manipulated by varying the ratio of PLA to PGA. Second, the physiologic compatibility of PLGA and its homopolymers PGA and PLA have been established for safe use in humans; these materials have a history of over 30 years in various human clinical applications. Finally, PLGA particles can be formulated in a variety of ways that improve biodistribution to target tissue by either passive or active targeting.

1. Synthetic Polymers

Non-biodegradable or biodegradable polymers may be used to form the microparticles. In the preferred embodiment, the microparticles are formed of a biodegradable polymer. Non-biodegradable polymers may be used for oral administration. In general, synthetic polymers are preferred, although natural polymers may be used and have equivalent or even better properties, especially some of the natural biopolymers which degrade by hydrolysis, such as some of the polyhydroxyalkanoates. Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

2. Biodegradable Polymers

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the microparticles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

3. Non-Degradable Polymers

Examples of preferred non-biodegradable polymers include ethylene vinyl acetate, poly(meth)acrylic acid, polyamides, copolymers and mixtures thereof.

In a preferred embodiment, PLGA is used as the biodegradable polymer.

C. Molecules to be Delivered

The disclosed microparticles can include one or more additional agents to be delivered to a targeted cell. Agents to be delivered include therapeutic, nutritional, diagnostic, and prophylactic compounds. Proteins, peptides, carbohydrates, polysaccharides, nucleic acid molecules, and organic molecules, as well as diagnostic agents, can be delivered. The preferred materials to be incorporated are immunosuppressive drugs or anti-inflammatory drugs. Therapeutic agents include antibiotics, antivirals (especially protease inhibitors alone or in combination with nucleosides for treatment of HIV or Hepatitis B or C), anti-parasites (helminths, protozoans), anti-cancer (referred to herein as “chemotherapeutics”, including cytotoxic drugs such as doxorubicin, cyclosporine, mitomycin C, cisplatin and carboplatin, BCNU, 5FU, methotrexate, adriamycin, camptothecin, and taxol), antibodies and bioactive fragments thereof (including humanized, single chain, and chimeric antibodies), antigen and vaccine formulations, peptide drugs, anti-inflammatories, nutraceuticals such as vitamins, and oligonucleotide drugs (including DNA, RNAs, antisense, aptamers, ribozymes, external guide sequences for ribonuclease P, and triplex forming agents).

Particularly preferred drugs to be delivered include anti-angiogenic agents, antiproliferative and chemotherapeutic agents such as rampamycin. Incorporated into microparticles, these agents may be used to treat cancer or eye diseases, or prevent restenosis following administration into the blood vessels.

Exemplary diagnostic materials include paramagnetic molecules, fluorescent compounds, magnetic molecules, and radionuclides.

D. Targeting Molecules

Targeting molecules can be proteins, peptides, nucleic acid molecules, saccharides or polysaccharides that bind to a receptor or other molecule on the surface of a targeted cell. In a preferred embodiment, the microparticle includes antibodies specific for proteins or lipids on the surface of cells expressing ILTs, preferably ILT2 and/or ILT4. Suitable antibodies include monoclonal, polyclonal, humanized, single chain antibodies, and fragments thereof that bind to a receptor on T cells an or dendritic cells. Preferred antibodies are those specific for pan-dendritic cell marker CD11c and antigen binding fragments thereof. Antibodies or ligands to the following dendritic cell markers can also be used: CD4, CD8, CCR7, CD1a, B7-B7-2, CD123, CD205, CD209, CD273, CD283, CD289, CMKLR-1, CXCR4, and combinations thereof. Chemokine receptors on dendritic cells can be target by chemokine ligands. For example for the CXCR4 receptor the ligand is CXCL12. DCs express CCR1, CCR5, CCR6 and ligands for them are CCL9, CCL3, and CCL20, respectively. For targeting T cells CD3, CD4, or CD8 can be targeted or any protein in the T cell receptor.

The degree of specificity can be modulated through the selection of the targeting molecule. For example, antibodies are very specific. These can be polyclonal, monoclonal, fragments, recombinant, or single chain, many of which are commercially available or readily obtained using standard techniques. Examples of molecules targeting extracellular matrix (“ECM”) include glycosaminoglycan (“GAG”) and collagen.

E. Pharmaceutical Acceptable Excipients

The microparticle compositions may be administered in combination with a physiologically or pharmaceutically acceptable carrier, excipient, or stabilizer. The term “pharmaceutically acceptable” means a non-toxic material that does not interfere with the effectiveness of the biological activity of the active ingredients. The term “pharmaceutically-acceptable carrier” means one or more compatible solid or liquid fillers, dilutants or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In the preferred embodiment, administration is by injection. Typical formulations for injection include a carrier such as sterile saline or a phosphate buffered saline. Viscosity modifying agents and preservatives are also frequently added.

Optional pharmaceutically acceptable excipients especially for enteral, topical and mucosal administration, include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE XL from GAF Chemical Corp).

Stabilizers are used to inhibit or retard decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-b-alanine, sodium N-lauryl-b-iminodipropionate, myristoamphoacetate, lauryl betaine and lauryl sulfobetaine.

If desired, the particles may also contain minor amount of nontoxic auxiliary substances such as wetting or emulsifying agents, dyes, pH buffering agents, or preservatives.

The particles may be complexed with other agents. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

The particles may be further coated. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Röhm Pharma, Darmstadt, Germany), zein, shellac, and polysaccharides. Additionally, the coating material may contain conventional carriers such as plasticizers, pigments, colorants, glidants, stabilization agents, pore formers and surfactants.

### I. Methods of Manufacture

In addition to the preferred method described in the examples for making a the disclosed microparticles, there may be applications where microparticles can be fabricated from different polymers using different methods.

A. Solvent Evaporation.

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microparticles. The resulting microparticles are washed with water and dried overnight in a lyophilizer. Microparticles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

B. Hot Melt Microencapsulation.

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microparticles are washed by decantation with petroleum ether to give a free-flowing powder. Microparticles with sizes between 0.5 to 1000 microns are obtained with this method. The external surfaces of spheres prepared with this technique are usually smooth and dense. This procedure is used to prepare microparticles made of polyesters and polyanhydrides. However, this method is limited to polymers with molecular weights between 1,000-50,000.

C. Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. Microparticles that range between 1-300 microns can be obtained by this procedure. The external morphology of spheres produced with this technique is highly dependent on the type of polymer used.

D. Spray-Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=24° C., outlet temperature=13-15° C., aspirator setting=15, pump setting=10 mL/minute, spray flow=600 Nl/hr, and nozzle diameter=0.5 mm. Microparticles ranging between 1-10 microns are obtained with a morphology which depends on the type of polymer used.

E. Hydrogel Microparticles.

Microparticles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath for twenty to thirty minutes in order to allow sufficient time for gelation to occur. Microparticle particle size is controlled by using various size extruders or varying either the nitrogen gas or polymer solution flow rates. Chitosan microparticles can be prepared by dissolving the polymer in acidic solution and crosslinking it with tripolyphosphate. Carboxymethyl cellulose (CMC) microparticles can be prepared by dissolving the polymer in acid solution and precipitating the microparticle with lead ions. In the case of negatively charged polymers (e.g., alginate, CMC), positively charged ligands (e.g., polylysine, polyethyleneimine) of different molecular weights can be ionically attached.

F. Surface Modification

There are two principle groups of molecules to be encapsulated or attached to the polymer, either directly or via a coupling molecule: targeting molecules, attachment molecules and therapeutic, nutritional, diagostic or prophylactic agents. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulthydryls and hydroxyls) present on the surface of the particle and present on the ligand to be attached. Functionality may be introduced into the particles in two ways. The first way is during the preparation of the microparticles, for example during the emulsion preparation of microparticles by incorporation of stablizers with functional chemical groups.

A second way for introducing ligands is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after prepartion. This second class also includes a process whereby amphiphilic molecules such as fatty acids, lipids or functional stabilizers may be passively adsorbed and adhered to the particle surface, thereby introducing functional end groups for tethering to ligands.

In the preferred embodiment, the surface is modified to insert amphiphilic polymers or surfactants that match the polymer phase HLB or hydrophile-lipophile balance, as demonstrated in the following example. HLBs range from 1 to 15. Surfactants with a low HLB are more lipophilic and thus tend to make a water in oil emulsion while those with a high HLB are more hydrophilic and tend to make an oil in water emulsion. Fatty acids and lipids have a low HLB below 10. After conjugation with a target group (such as hydrophilic avidin), HLB increases above 10. This conjugate is used in emulsion preparation. Any amphiphilic polymer with an HLB in the range 1-10, more preferably between 1 and 6, most preferably between 1 and up to 5, can be used. This includes all lipids, fatty acids and detergents.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

### IV. Methods of Use

The disclosed compositions can be used for the treatment of one or more symptoms of an autoimmune disease, inflammatory disorder, or graft rejection by administering an effective amount of a microparticle containing HLA-G dimers and a targeting moiety to decrease the level of expression of MHC class II, CD80, CD86 or a combination thereof in dendritic cells. In other embodiments the double-coated microparticles down-regulate DC activation and function and thereby treat one or more symptoms of an inflammatory disorder or disease or one or more symptoms of an autoimmune disorder or disease.

Signaling events downstream of the ILT receptors and their functional impact on DC activation/maturation are incompletely understood. Engagement of ILT4 by HLA-G ligand results in recruitment of both SHP-1 and SHP-2 phosphatases and involves the IL-6-STAT3 pathway. Analysis of human DC and experiments with murine ILT4-positive DC suggest that one of the major targets of the HLA-G and ILT4 interaction on DC is MHC class II molecules. During maturation, DC increase their surface expression of MHC class II molecules by several fold. This increase is accompanied by a dramatic change in localization of MHC class II molecules, which are abundant in endosomal structures in immature DC but are located mostly on the plasma membrane in mature DC. The control of MHC class II molecule synthesis and degradation, trafficking, and peptide loading represent key mechanisms in antigen presentation by DC. Recently, it was discovered that the IL-6-STAT3 pathway controls the intracellular MHC class II αβ dimer level through cathepsin S activity in DC.

Although IL-6 is involved in the development of mature T and B cell responses, it does not have only pro-inflammatory properties. It was recently demonstrated that forced activation of cytokine IL-6 in DC resulted in the development of tolerogenic DC. IL-6 knockout mice had an increased number of mature DC, indicating that IL-6 blocks DC maturation in vivo. The engagement of ILT4 on DC by certain isoforms of HLA-G results in increasing the transcriptional and protein levels of IL-6 and conferring DC with tolerogenic properties. The signaling pathway of IL-6 leads to the activation of STAT3 and STAT1. IL-6 activates STAT3 exclusively; when IL-6 levels were raised 10 to 100 fold, STAT1 activation was noted as well. It has been shown that the treatment of ILT4-positive DC with HLA-G1 tetramer and HLA-G5 dimer induced phosphorylation of STAT3. Additional stimulation of DC with lipopolysaccharide (LPS) increases the levels of STAT3 protein and enhances its phosphorylation. In contrast, no activation of STAT1 was detected. Experiments with knockdown of tyrosine phosphatases determined that SHP-2 was a key molecule involved in the increase of IL-6 by the HLA-G/ILT4 interaction on DC during the maturation process that was mediated by LPS signaling. Since the HLA-G/ILT4 interaction on DC especially targets MHC class II genes and does not affect the expression of MHC class I molecules, it is most likely that in ILT4-positive DC, SHP-2 modulates the NF-κB pathway in a MAP kinase-independent fashion in induction of IL-6. The exact molecular mechanisms of the induction of negative regulators of TLR4 signaling remain to be determined.

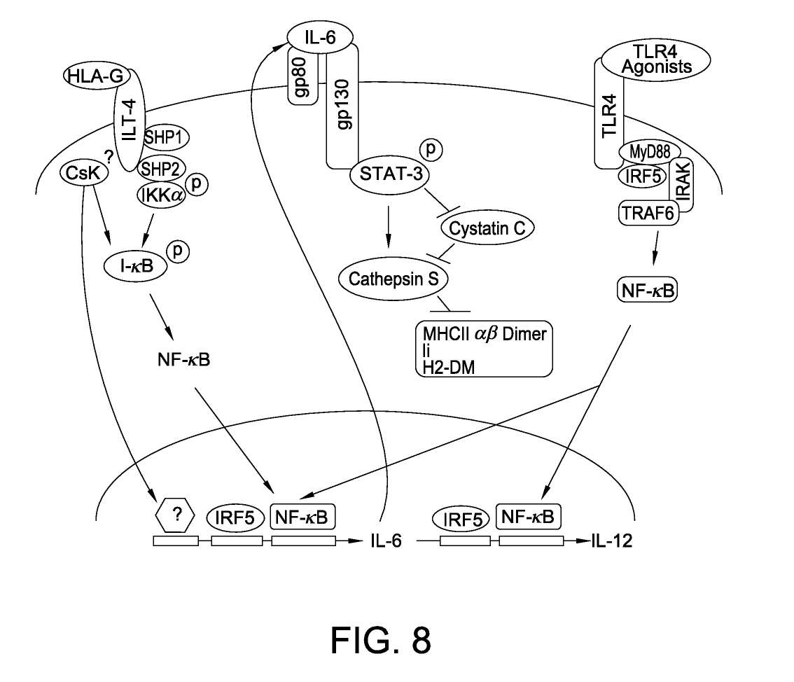

The mechanism described in FIG. 8 can be applied to control the maturation/activation of DC via HLA-G/ILT4 in the absence of a strong inflammatory response and during a moderate signal through TLR4. This situation could be similar or equivalent to a normal pregnancy or surgical procedure with tissue or organ transplantation. However, upon receiving a strong activated signal associated with a pathogen or inflammation, ILT4-positive DC will most likely force a robust rise in IL-6 levels, which will result in activation of STAT3 and STAT1, conferring DC with immune-stimulating properties.

All DCs have a capacity for initiating tolerance or immunity; the distinction depends on the maturation or activation state of the DCs (26, 27). The presence of powerful inhibitory receptors on DCs is indicative of their potential to control the activation or maturation of DCs and confer tolerogenic capacity. HLA-G5 dimer and HLA-G1 tetrameric complexes have a similar capacity to induce an ILT-mediated inhibitory signal and modulation of DC activation and maturation. In contrast, HLA-05 monomer neither triggers an ILT inhibitory signal nor modulates ILT4-positive DCs in vitro and in vivo. The precise role of different isoforms of HLA-G in the modulation of DCs is dependent on their concentration and conformation, affecting binding to a specific receptor. At least in vitro, the increasing concentration of HLA-G5 monomeric form significantly enhanced the formation of HLA-G5 dimer. Thus, in this situation, the ILT-mediated inhibitory signal occurs via the HLA-G5 dimer form, since a high concentration of purified HLA-G5 monomer did not trigger the ILT-mediated signal. It is most likely that the monomeric form of HLA-G5 plays an important function involving the control of angiogenesis. Monomeric HLA-G5 has been shown to bind an activating receptor, KIR2DL4, on human resting NK cells and trigger the expression of a set of chemokines and cytokines driving a pro-inflammatory/pro-angiogenic response.

A. Inflammatory and Autoimmune Diseases

The double-coated microparticles can be used to treat one or more symptoms of an inflammatory disease or disorder. Representative inflammatory diseases or disorders that can be treated with the microparticles to reduce, inhibit or mitigate one or more symptoms include, but are not limited to, autoimmune diseases or disorders including rheumatoid arthritis, systemic lupus erythematosus, alopecia greata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

An effective amount of the disclosed microparticles can be administered to a subject in need thereof to modulate activity of immune cells that express ILTs, in particular ILT2 and/or ILT4.

B. Graft Rejection

The disclosed microparticles can also be used to reduce or delay graft rejection for example allogeneic skin graft rejection relative to a control. One embodiment provides a method for inducing and/or maintaining transplantation tolerance by administering to a subject an effective amount of a the disclosed double-coated microparticles to own-regulate DC activation and function in the subject. The induction or maintenance of transplantation tolerance can be determined relative to a control.

### EXAMPLES

### Example 1

### HLA-G Tetramer Coated Microparticles

Mice.

C57BL/6, and B6.C-H-2^{bm12} (bm12) were purchased from Jackson Laboratory, Bar Harbor, Me. ILT4-transgenic mice have been described previously (Ristich, V., Eur J Immunol, 35:1133-1142 (2005)). The use of animals for this study was approved by the animal care committee of the Medical College of Georgia.

Generation of HLA-G5 Monomer, HLA-G5 Dimer, and HLA-G1 Tetrameric Complexes Coupled to Polystyrene Microspheres.

HLA-G5 was PCR-amplified from the JEG-3 choriocarcinoma cell line and the cDNA subcloned into the pcDNA3 expression vector. HLA-negative cells 721.221 were stable transfected with HLA-G5 expression construct. HLA-G5 protein was obtained from supernatant of 721.221/HLA-G5 cells using Hi Trap NHS activated HP columns (Amersham Biosciences, Piscataway, N.J.) coated with W6/32 mAb. The columns were blocked with 100 mM ethanolamine, pH 9.0. After washing, 100 ml of supernatant of 721.221/HLA-G5 cells was applied onto the column overnight at 4° C. After washing in PBS, bound protein was eluted with 0.1 M glycine buffer pH 11.0 neutralized with Tris 1M buffer solution, pH 7.5. The presence of HLA-G5 monomer in the eluted fraction was confirmed using ELISA and immunoblot analysis. Additional incubation of HLA-G5 monomer at 4° C. for 7 d significantly promoted HLA-G5 dimer formation, and this was the major source for the purification of HLA-G5 dimer. HLA-G1 tetrameric complexes have been generated as described previously (Ristich, V., et al., Eur J Immunol, 35:1133-1142 (2005)). Tetramer microspheres were generated using 5.3-mm diameter polystyrene microspheres (Interfacial Dynamics, Portland, Oreg.). Subsequently, tetramers were adsorbed onto the microspheres by incubating overnight at 4° C. with purified tetramers. NFAT-GFP reporter cells expressing the ILT2-PILRβ chimera described previously (Shiraishi M, et al. J Mal Chem, 281: 10439-10447 (2006)).

Antibodies and Flow Cytometry Analysis.

DCs were stained with mAbs anti-CD11c-APC (HL3, hamster IgG), anti-CD11b-FITC (M1/70, rat IgG2b), anti-MHC class II-FITC (M5/114.15.2, rat IgG2b), anti-CD80 (16-10A1, hamster IgG), anti-CD86 (GL1, rat IgG2a), or purified anti-ILT4 (42D1, rat IgG2a) followed by FITC-labeled goat anti-rat IgG. All primary and secondary reagents were purchased from BD-Pharmingen (San Diego, Calif.) or from eBioscience unless otherwise specified. Cells were incubated with primary antibodies in PBS containing 2% BSA for 30 min at 4° C. and washed twice with PBS containing 2% BSA. When necessary, conjugated secondary antibodies were used. In some experiments DCs were treated with anti-IL-6 neutralizing antibody MP5-20F3 (eBioscience). Intracellular staining of cytokines was performed as described previously (21). A BD Biosciences FACSCalibur (Mountain View, Calif.) was used for data acquisition and CellQuest software was used for analysis.

Statistical Analysis

Where applicable, values were compared by the Mann-Whitney U test using Stata 7.0 Software.

Results

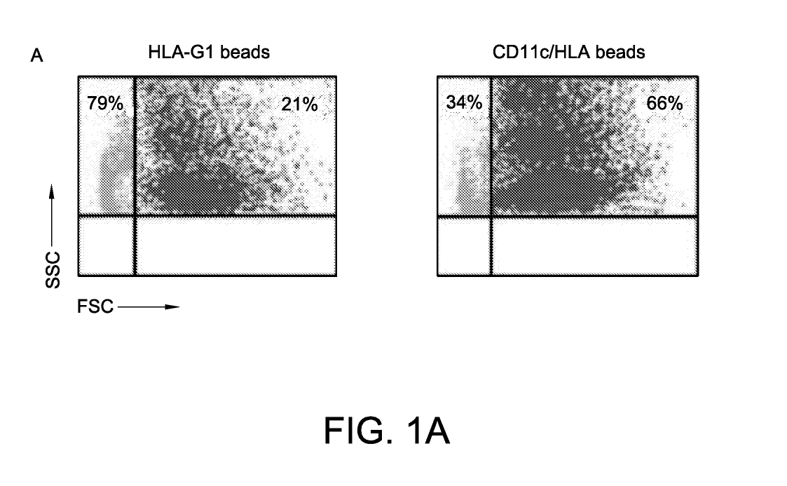

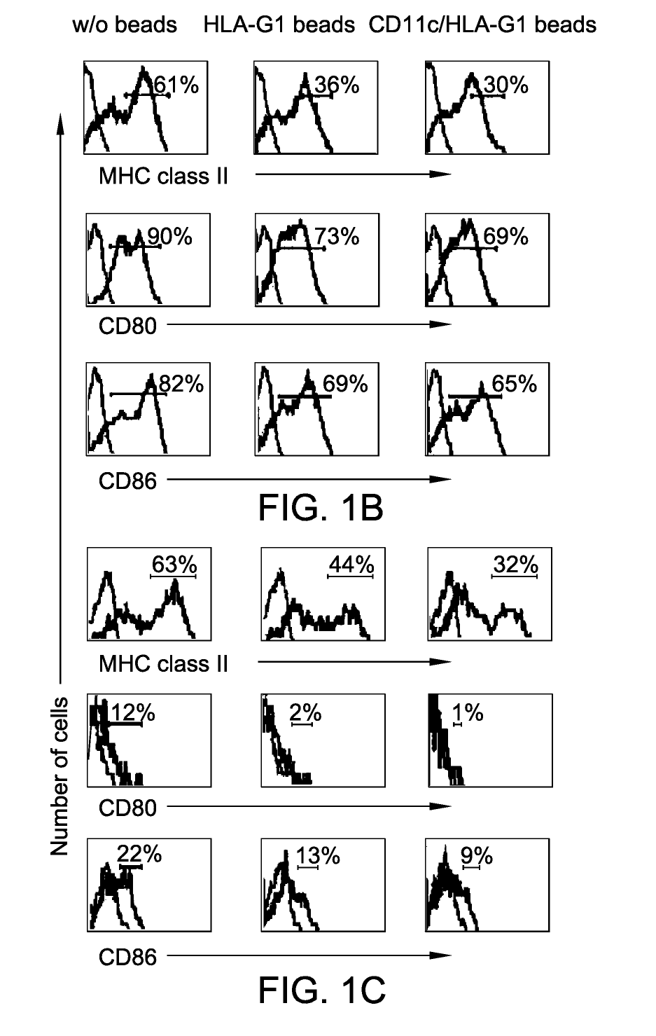

Bone marrow-derived DC (BMDC) were prepared from ILT4 transgenic mice as described previously (Ristich, V, et al., Eur J Immunol, 35:1133-1142 (2005)). 5×10⁶ microparticles (Invitrogen, Carlsbad, Calif.) were coated with HLA-G1 tetrameric complexes (Liang, S., et al., Proc Nall Acad Sci USA, 105:8357-8362 (2008)) or with both a purified anti-CD11c mAb (N418, hamster IgG, eBioscience, San Diego, Calif.) and HLA-G1 tetramer, and added to DC for 3 h. Cells were analyzed by flow cytometry. Numbers indicate the percentage of microparticles bound to ILT4-positive DC (top, right quadrant) and unbound (top, left quadrant) microparticles (FIG. 1A). Data shown are from one of four independent experiments and show that double-coated microparticles increase binding to ILT4-positive DC in vitro and in vivo and enhance their inhibitory effect.

FIG. 1B shows the effect of single (HLA-G1-coated) and double (HLA-G1 and anti-CD11c mAb-coated) microparticles on activation/maturation of ILT4-positive DC in vitro. Immature BMDC from ILT4 mice (1×10⁶) were treated for 3 h with the same amount of the indicated microparticles, and cells were stimulated for an additional 18 h with a titrated concentration of LPS (Sigma-Aldrich, St. Louis, Mo.; 100 ng/ml of LPS as shown). Cells were stained with anti-MHC class II mAb (M5/114.15.2, rat IgG2b, BD Pharmingen, San Diego, A), anti-CD80 mAb (16-10A1, hamster IgG), and anti-CD86 mAb (GL1, rat IgG2a, both mAbs from e-Bioscience). Histograms shown were gated on the CD11c+ population. Numbers indicate percentage of total gated cells falling into selected quadrants. Numbers for MHC class II represent by population of cells, which highly express these molecules. Data are representative of four independent experiments.

FIG. 1C shows the effect of single (HLA-G1-coated) and double (HLA-Gland anti-CD11c mAb-coated) microparticles on activation/maturation of ILT4-positive DC in vivo induced by allogeneic skin transplant. Draining lymph node cells isolated from ILT4 transgenic mice (H-2^{b}) grafted with allogeneic skin transplant from a MHC class II-mismatch donor (B6.C-H-2bm12, H-2^{b}) were analyzed on day 3 for the expression of MHC class II and costimulatory molecules CD80 and CD86. One day before grafting, mice received single- or doubled-coated microparticles as indicated. Histograms shown were gated on the CD11c⁺ population. Numbers indicate percentage of total gated cells falling into selected quadrants. Numbers for MHC class II represent by population of cells, which highly express these molecules. Data are representative of three independent experiments.

### Example 2

### HLA-G5 Dimer and HLA-G1 Tetramer Induce Strong ILT-Mediated Signaling In Vitro

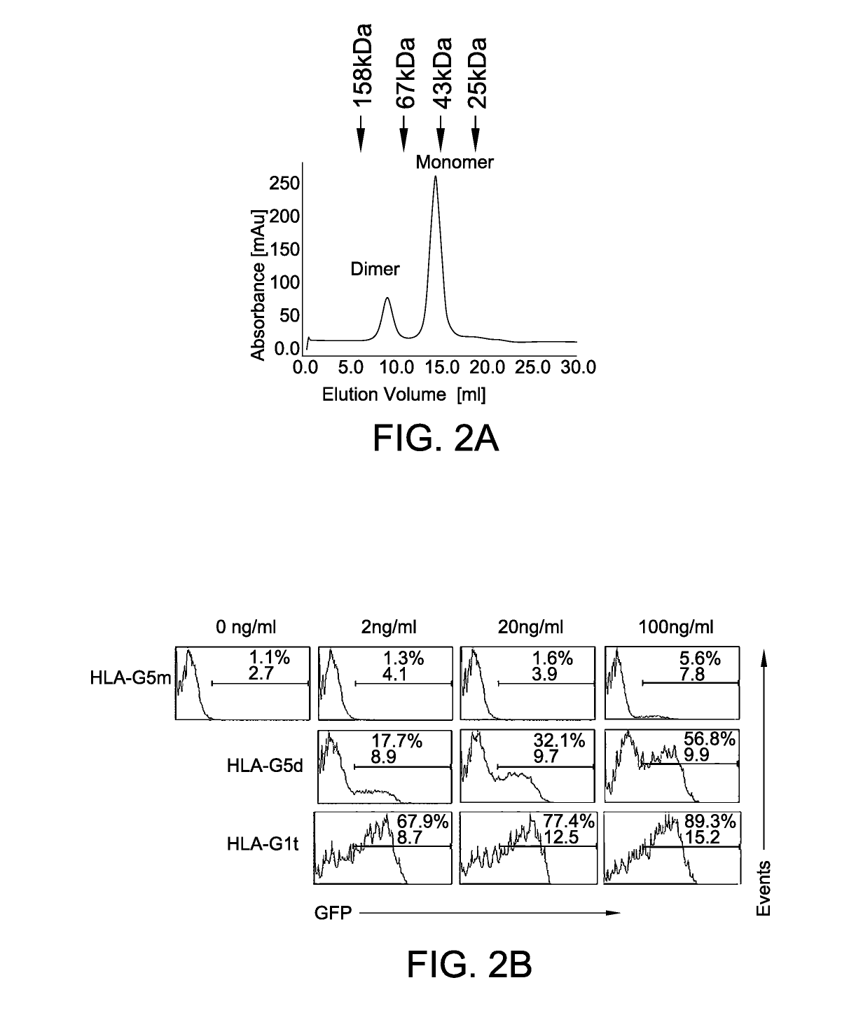

The formation and induction of efficiency of ILT-mediated signaling by HLA-G5 monomer (HLA-G5m) and HLA-G5 dimer (HLA-G5d) was analyzed using supernatant from an HLA-negative cell line transfected with HLA-G5. The monomers and dimers were produced as described in Example 1. HLA-G5 monomer was purified, and the gel filtration chromatogram showed a single peak corresponding to the expected molecular mass (MM) of 49 kDa (data not shown). It has been demonstrated that several factors, including the concentration of monomer, low temperature, or dithiothreitol, affect the dimerization of HLA-G. Size-exclusion chromatography of HLA-G5m incubated at 4° C. for 7 d showed the presence of two peaks, one corresponding to the MM of 49 kDa and the other corresponding to approximately twice that (FIG. 2A). Using an HLA-G-specific mAb, the presence of two bands were detected under nonreducing conditions, one of ≈37 kDa, the expected MM of the heavy chain of the soluble form of HLA-G5, and the other ≈74 kDa, the expected MM of dimerized HLA-G5 heavy chain. However, only a small fraction (<10%) of the entire pool of HLA-G5 heavy chains exists in the dimerized form. The analysis of avidity of the different isoforms of HLA-G on ILT2-mediated signaling using an NFAT-GFP reporter system showed minor stimulation of the reporter cells by HLA-G5m (FIG. 2B). Even at a high concentration of 100 ng/ml of HLA-G5m, only 5.6%±0.86%, mean±SD, of total cells were GFP-positive, with a mean fluorescent intensity (MFI) of 7.8±0.9, FIG. 1C). In contrast, HLA-G5d remarkably enhanced ILT2-mediated signaling at a much lower concentration (2 ng/ml), and at 100 ng/ml 56.8%±7.2% cells were GFP-positive (MFI=9.9±1.1), suggesting that the HLA-G5d form has more efficient signaling and similar efficacy to HLA-G1 tetrameric complexes (HLA-G1t) (FIG. 1C). The results indicate that the dimer form of HLA-G5 and the tetrameric complexes of HLA-G1 are potent to induce the most efficient ILT-mediated inhibitory signaling.

### Example 3

### Arrest of Maturation/Activation of ILT4-Positive DCs In Vivo by Different Isoforms of HLA-G

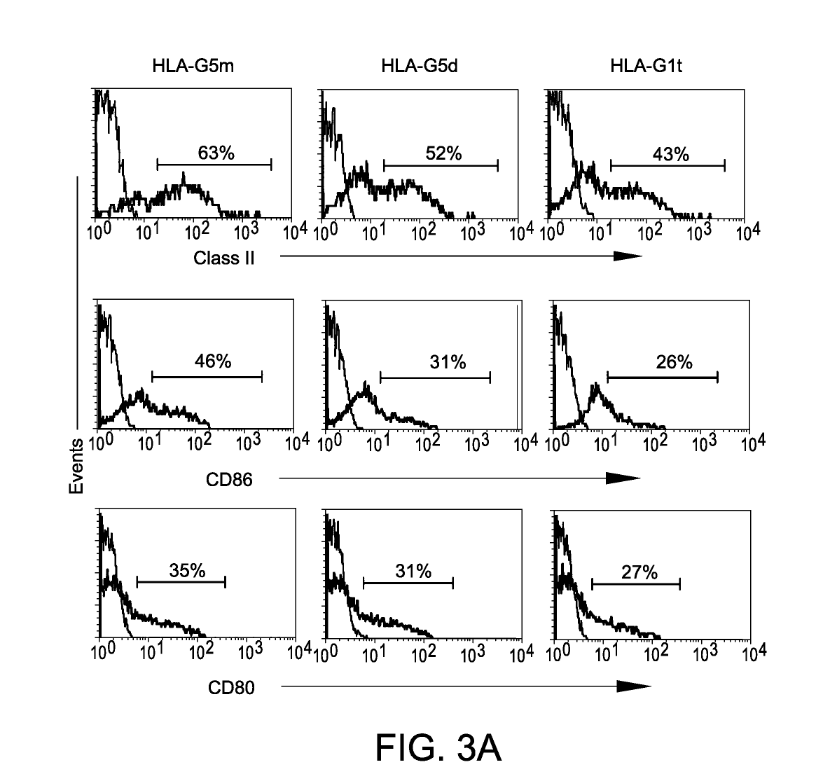

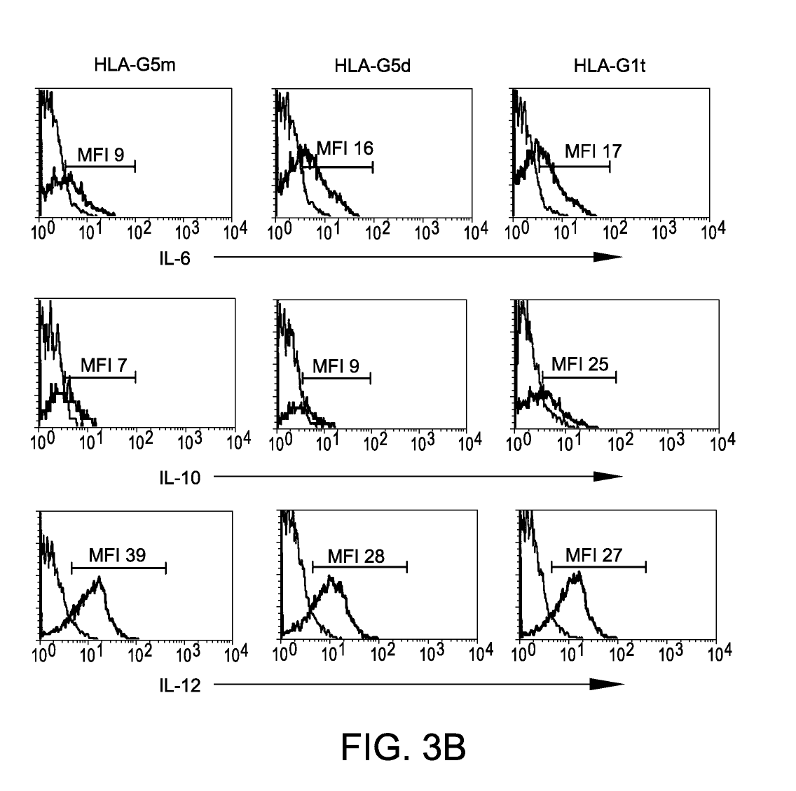

To examine the effect of different isoforms of HLA-G on the activation/maturation of ILT4-positive DCs in vivo, the number of activated/mature and immature DCs in draining lymph nodes and in spleens from recipient ILT4 transgenic mice after allogeneic skin transplantation from the MHC class II-disparate mutant B6.C-H-2^{bm12} (bm12) donor mice were analyzed at different time points. Transgenic mice expressing ILT4 receptor exclusively on DCs have been described previously. In addition, analysis of the key cytokines (IL-6, IL-10, and IL-12) involved in maturation/activation of DCs was evaluated by intracellular staining with cytokine-specific mAbs and flow cytometry. The number of activated/mature DCs that expressed high levels of MHC class II molecules and CD86 with elevated levels of IL-12 was decreased in lymph nodes from ILT4 mice targeted with HLA-G5d and HLA-G1t (FIGS. 3A and B). In contrast, ILT4 mice treated with HLA-G5m had more activated/mature DCs and a similar phenotype to the DCs from ILT4 mice untreated with HLA-G. Moreover, it was observed that immature DCs from mice targeted with HLA-G1t and with HLA-G5d, have enhanced expression of IL-6, demonstrated by the increased numbers of cells and capacity to secrete IL-6 (6B). These results further strengthen the hypothesis that different isoforms of HLA-G have distinct roles in suppression of activation/maturation of DCs through the ILT4 receptor.

### Example 4

### Engagement of ILT4 by HLA-G Ligands Increases Transcriptional Levels of IL-6 in DCs

RNA Isolation and DNA Microarray Analysis.

Total RNA from BMDCs treated with HLA-G ligands was isolated with RNA STAT-60™ Kits (Tel-Test, Friendswood, Tex.). BMDCs without treatment were used as a control and processed accordingly. cDNA probe was generated with GEArray TrueLabeling-RT kit (SuperArray Bioscience, Frederick, Md.). Following denaturation, the cDNA probe was hybridized to murine Dendritic & Antigen Presenting Cell Gene Array at 60° C. for 16 h. Data were analyzed by the Image Quant 1.2 Software (Amersham Biosciences) with STORM™ 840 Gel and Blot Imaging System (Amersham Biosciences). Signal for each transcript was normalized by comparing to the housekeeping gene GAPDH.

RT-PCR Analysis for Quantitation of IL-6 mRNA.

The relative amount of IL-6 mRNA was assessed by semiquantitative RT-PCR followed by Southern blot of the RT-PCR products. RNA was isolated and cDNA synthesized as described above. Subsequent PCR analysis was performed using the manufacturer's suggested protocol. Primer oligonucleotides were synthesized by Integrated DNA Technologies: IL-6 5′ primer, ATGAAGTTCCTCTCTGCAAGAGAC (SEQ ID NO:1) and IL-6 3′ primer, CACTAGGTT TGCCGAGTAGATCTC (SEQ ID NO:2); β-actin 5′primer, GTGGGGCGCCCCAGGCACCA (SEQ ID NO:3) and β-actin 3′ primer CTCCTTAATGTCACGCACGATTTC (SEQ ID NO:4).

Results

To investigate the role of certain isoforms of HLA-G on the regulation of gene expression in ILT4-positive bone marrow-derived DCs (BMDCs), the gene expression profiles of cells exposed to HLA-G ligands were analyzed at different times using the mouse dendritic and antigen-presenting cell Gene Array System (SuperArray Bioscience).

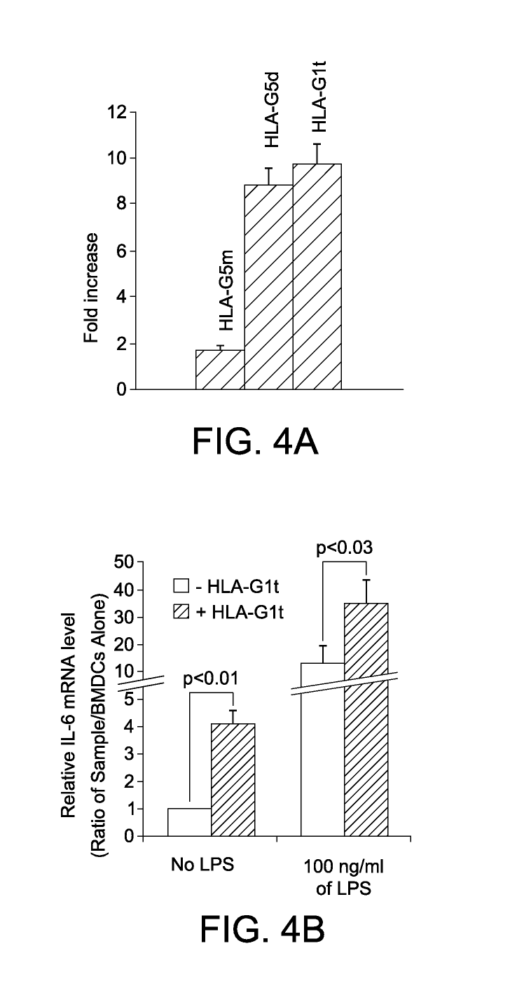

The comparison between HLA-G5m-treated and non-treated ILT4-positive DCs shows that the expression levels of most transcripts were similar. However, treatment of DCs with HLA-G5d or HLA-G1t affected several genes, the majority of which were down regulated. These genes include chemokine ligand 3 (CCL3), myristoylated alanine rich protein kinase C substrate (MARCKS), interferon-induced with tetratricopeptide repeats 1 (IFIT1), and histocompatibility class II locus DMa (H2-DMa). On the other hand, the expression levels of a very limited number of genes were up regulated. These genes include guanylate nucleotide binding protein 3 (GBP3) and CD36. The most frequent transcript was identified as IL-6 (8.9-fold and 9.3-fold increase by HLA-G5d and HLA-G1t, respectively; FIG. 4A). RT-PCR analysis of ILT4-positive BMDCs revealed that IL-6 transcription can be detected in HLA-G1t-treated cells without stimulation with LPS but was considerably increased in the treated cells following activation/maturation with LPS (FIG. 4B). The same results were obtained with ILT4 BMDCs treated with HLA-G5d (data not shown). These data suggest that the transcriptional level of IL-6 on ILT4-positive DCs is mediated by particular isoforms of HLA-G and can be modified by LPS signaling.

### Example 5

### IL-6 Neutralization Abolishes Inhibition of the Expression of MHC Class II Molecules on DCs Mediated by HLA-G and ILT4

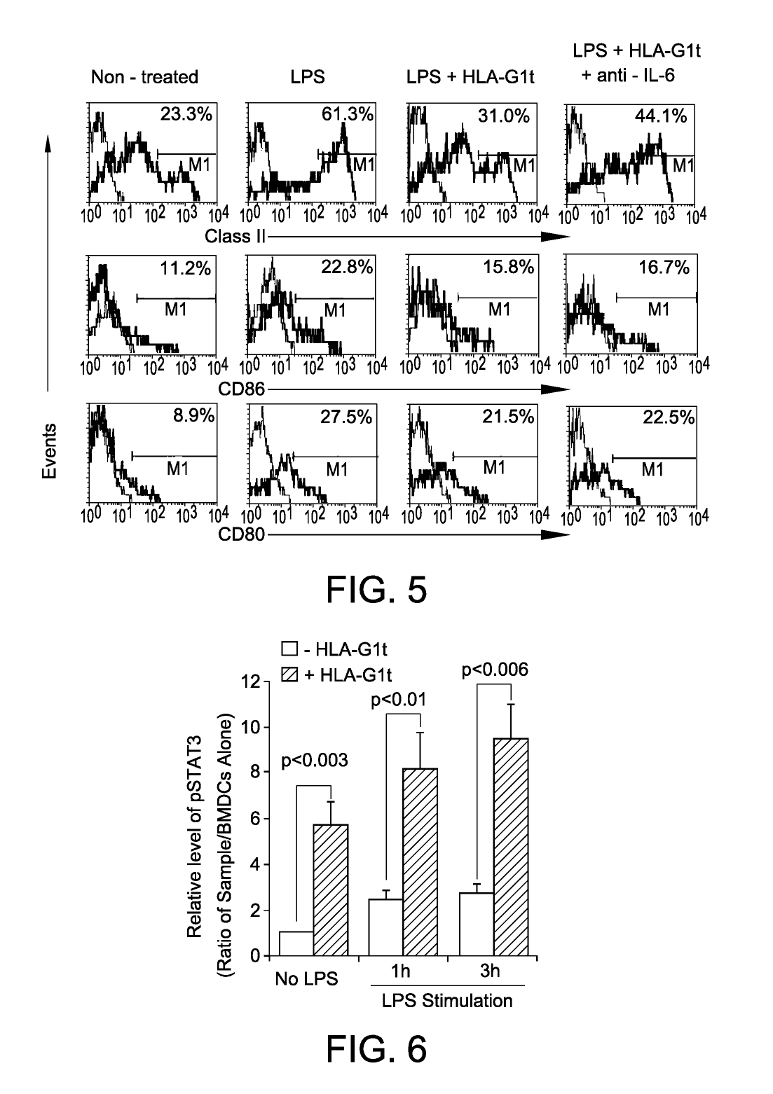

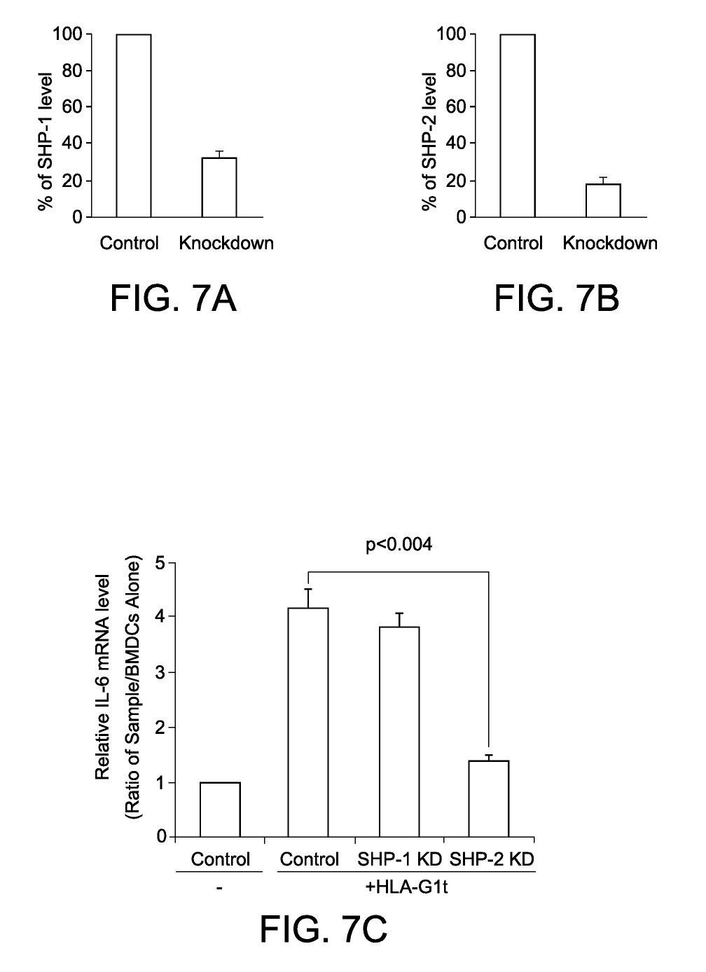

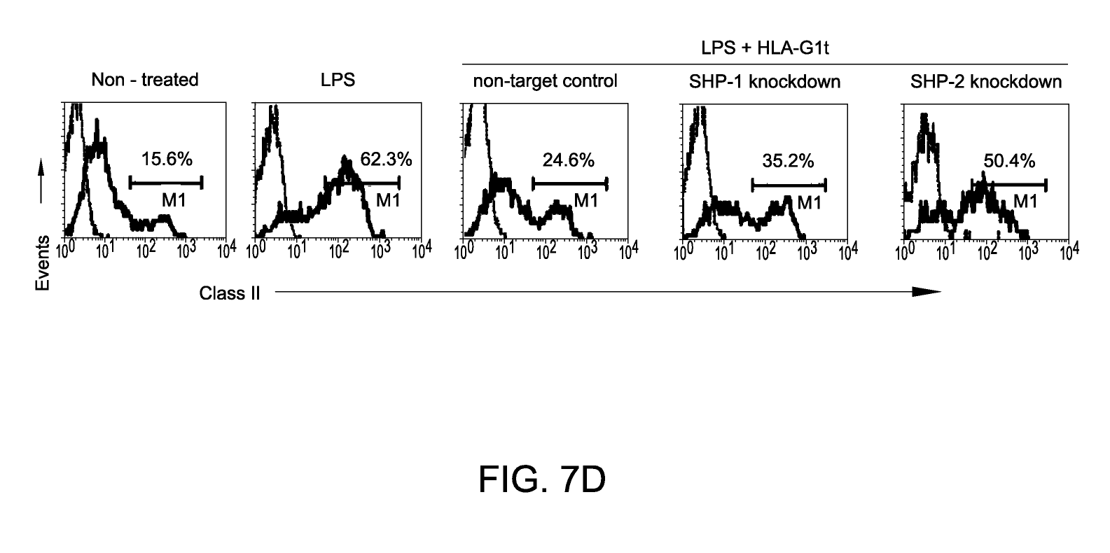

Bone marrow-derived immature myeloid ILT4-positive DCs were subjected to LPS stimulation for 18 hr, which resulted in increased expression of MHC class II molecules (increased number of MHC class II-positive cells from 23.3%±2.6% to 61.3%±8.9%, mean±SD, p<0.002; upper panels, SI FIG. 7). Treatment of ILT4-positive DCs with HLA-G following stimulation with LPS decreases cell surface expression of MHC class II molecules (number of MHC class II-positive cells decreases after treatment with HLA-G1t from 61.3%±8.9% to 31.0%±4.5%, p<0.006, FIG. 5). Additional treatment of ILT4-positive DCs with anti-IL-6 neutralizing antibody increased the number of MHC II-positive cells from 31.0%±4.5% to 44.1%±6.5%, p<0.02, SI FIG. 7) and abolished the HLA-G1t-mediated inhibition of expression of MHC class II molecules on ILT4-positive DCs. A similar abolishing effect of neutralizing anti-IL-6 antibody was found on ILT4-positive DCs treated with HLA-G5d (data not shown). The treatment of DCs with neutralizing anti-IL-6 antibody has no effect on the number of cells that are positive for CD86 (middle panels, SI FIG. 5). These data suggested that IL-6 plays a major role in the down-regulation of expression of MHC class II molecules by HLA-G and ILT4 on DCs.

### Example 6

### The Arrest of Maturation/Activation of ILT-4-Positive DCs by HLA-G is Correlated with an Increase in STAT3 Activation, but not in STAT1

Immunoprecipitation and Western Blot Analysis.

The pellets from 5×10⁶ untreated DCs and DCs treated with HLA-G ligands and LPS were lysed in 1 ml RIPA buffer and precleared several times with Sepharose-coupled protein A (50% wt/vol slurry). Proteins were immunoprecipitated with saturating amounts of anti-ILT4 mAb and protein G Sepharose (Amersham Biosciences). Immunoprecipitation experiments were performed using several ILT4-specific antibodies, including polyclonal anti-ILT4 (C-12), anti-ILT4 (N-14), anti-ILT4 (P-14), anti-ILT (H-300), and anti-ILT4 (42D1) mAb (all from Santa Cruz Biotechnology, Santa Cruz, Calif.). Immunoblotting was performed with anti-phosphotyrosine antibody (4010; Upstate, Temecula, Calif.). The immunoblot was reprobed with anti-SHP-1 and anti-SHP-2 antibodies (both Upstate), and anti-Csk antibody (Santa Cruz Biotechnology). Signals were developed using the enhanced chemoluminescence (ECL) system (Amersham Biosciences). In some cases, densitometric scans of bands were obtained to determine the extent of ILT4-mediated signal inhibition.

Results

To investigate the relationship between an increased level of IL-6 and STAT3 activation, the phosphorylation of STAT3 molecules in ILT4-positive HLA-G-treated DCs were analyzed. The levels of STAT3 activation in ILT4-positive BMDCs treated with HLA-G1t for 3 h with and without stimulation with 100 ng/ml of LPS for the indicated time. ILT4-positive BMDCs (5×10⁶) were treated for 3 h with 50 ng/ml of HLA-G1t or were left untreated. DCs were stimulated for an additional 1 h with 100 ng/ml of LPS or left unstimulated. Cells were lysed and proteins were immunoprecipitated with anti-STAT3 or anti-STAT1 antibodies. Immunoprecipitates were analyzed on Western blot for STAT proteins and reprobed with anti-phosphotyrosine antibody 4010. Proteins were visualized by enhanced chemiluminescence. The treatment of ILT4-positive DCs with HLA-G1t induces phosphorylation of STAT3 molecules (FIG. 6). Additional stimulation of DCs with LPS increases the levels of STAT3 protein and enhances phosphorylation of STAT3 molecules (FIG. 3). No activation of STAT1 was detected in HLA-G1t-treated DCs without LPS stimulation and stimulated with LPS. Similar data were observed in ILT4 DCs treated with HLA-G5d (data not shown). Together, these results demonstrate direct involvement of STAT3 activation in HLA-G-mediated arrest of maturation/activation of ILT4 DCs.

### Example 7

### Engagement of ILT4 Receptors by HLA-G on DCs Results in Phosphorylation of ILT4 and Recruitment of SHP-1 and SHP-2 Protein Tyrosine Phosphatases

Using different isoforms of HLA-G, it was determined that ILT4 becomes phosphorylated after engagement of ILT4 receptor with HLA-G5d or HLA-G1t and recruits both SHP-1 and SHP-2 phosphatases. Recently, it has been shown that C-terminal Src kinase (Csk) binds ILT2 and potentially regulates its function (24). In addition, it has been shown that Csk plays an important role in LPS-induced signal transduction and IL-6 production in myeloid cells (25). However, the presence of Csk in ILT4 immunoprecipitates from DCs was not detect (data not shown). No phosphorylation of ILT4 receptor was determined on ILT4-positive DCs treated with HLA-G5m (data not shown).

### Example 8

### Down-Regulation of SHP-2 Gene Expression, but not SHP-1, Diminishes Inhibitory Effect of HLA-G and ILT4 in DCs

shRNA-Targeted Protein Knockdown.

pLKO.1-puro lentiviral vectors expressing non-target control shRNA (SHC002) or one of 5 different SHP-1 shRNAs (TRCN0000028964, TRCN0000028965, TRCN0000028966, TRCN0000028967, TRCN0000028968) or one of 5 different SHP-2 shRNAs (TRCN0000029874, TRCN0000029875, TRCN0000029876, TRCN0000029877, TRCN0000029878) were purchased (Sigma-Aldrich, St. Louis, Mo.). The lentiviral packaging plasmid pCMV delta R8.2 and envelope plasmid pMD2.G were from Addgene (Cambridge, Mass.). 293 FT cells (Invitrogen, Carlsbad, Calif.) were co-transfected with shRNA lentiviral plasmid along with pCMV delta R8.2 and pMD2.G at a 10:1 ratio for the production of lentiviral particles. Transfections were carried out using TransIT-LT1 (Mirus Bio, Madison, Wis.), and virus was harvested 72 h post-transfection. BMDCs were infected with lentiviral particles twice (24 h, 48 h) and the knockdown efficiency of the targeted proteins was assessed by Western blot.

Results

To determine the role of SHPs recruited to phosphorylated ILT4 receptor after engagement with HLA-G on the level of IL-6 and on the differentiation of ILT4-positive DCs, analyses with down-regulated gene expression of these molecules was performed using short hairpin RNA (shRNA) knockdown technology. Using lentiviral transduction particles containing shRNAs for the target genes, we were able to down-regulate ≈70% of SHP-1 and ≈80% of SHP-2 gene expression in DCs, as detected by immunoblot analyses (FIGS. 7A and B). Knockdown of SHP-1 slightly reduces mRNA levels of IL-6 compared to non-target control shRNA on ILT4-positive DCs treated with HLA-G1t (FIG. 7C). In contrast, knockdown of SHP-2 resulted in 65% reduction of mRNA levels of IL-6 in cells treated with HLA-G1t, suggesting that SHP-2 plays a critical role for up-regulation of IL-6 in ILT4-positive DCs. In addition, we determined that down-regulation of SHP-1 has very little effect on alteration of the reduced level of expression of MHC class II molecules mediated by HLA-G1t-treatment of ILT4-positive DCs (FIG. 7D). However, down-regulation of SHP-2 increases the level of MHC class II molecules on ILT4-positive DCs treated with HLA-G1t, suggesting that SHP-2 is a key molecule in diminishing the inhibitory effect of HLA-G and ILT4 in DCs. Based on these data, we propose a model on the potential role of SHP-2 and the IL-6-STAT3 pathway in down-regulation of expression of MHC class II molecules and control of DC differentiation by HLA-G and ILT4 (FIG. 8).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A microparticle comprising
an HLA-G dimer displayed on the surface of the microparticle; and
a targeting moiety displayed on the surface of the microparticle for targeting delivery of the microparticle to an immune cell expressing ILT2 or ILT4.

2. The microparticle of claim 1 wherein the microparticle is biodegradable.

3. The microparticle of claim 1 wherein the targeting moiety comprises an antibody or antigen binding fragment thereof or a ligand specific for receptors on dendritic cells.

4. The microparticle of claim 3 wherein the antibody or antigen binding fragment thereof or ligand is specific for CD11c.

5. The microparticle of claim 1 wherein the microparticle has a diameter of 0.5 to 1000 microns.

6. The microparticle of claim 1 wherein the microparticle has a diameter of 50 nm to 500 nanometers.

7. The microparticle of claim 1 wherein the microparticle has a high density of ligands specific for dendritic cells or coupling agents.

8. The microparticle of claim 1 wherein the ligands specific for dendritic cells are present in the range of 1,000 to 10,000,000, more ligands per square micron of microparticle surface area.

9. The microparticle of claim 1 wherein the HLA-G dimer comprises HLA-G1.

10. The microparticle of claim 1 wherein the HLA-G dimer comprises HLA-G5.

11. The microparticle of claim 1 wherein the microparticle comprises HLA-G1 and HLA-G5.

12. The microparticle of claim 1 further comprising one or more therapeutic agents.

13. The microparticle of claim 12 wherein the one or more therapeutic agents include anti-inflammatory agents.

14. The microparticle of claim 1 further comprising a physiologically or pharmaceutically acceptable carrier, excipient, or stabilizer.

15. The microparticle of claim 1 for use in therapy.

16. A method for delaying transplant rejection in a subject comprising administering an effective amount of the microparticle according to claim 1 to the subject to enhance signal transduction through ILT2 or ILT4 on immune cells relative to a control.

17. A method for treating one or more symptoms of an inflammatory disorder n in a subject comprising administering an effective amount of the microparticle according to claim 1 to the subject t to enhance signal transduction through ILT2 or ILT4 on immune cells relative to a control.

18. A method for treating one or more symptoms of an autoimmune disorder in a subject comprising administering an effective amount of the microparticle according to claim 1 to the subject to enhance signal transduction through ILT2 or ILT4 on immune cells relative to a control.

19. The method of claim 16 wherein the immune cells are T cells or dendritic cells.

20. A method for decreasing the level of expression of MHC class II, CD80, CD86 or a combination thereof in dendritic cells comprising contacting the dendritic cells with an effective amount of the microparticle according to claim 1 to the subject to enhance signal transduction through ILT2 or ILT4 on the dendritic cells relative to a control.
